SEQUENCE LISTING (31 sequences)

SEQ ID NO: 1 - DNA, 20 bp, Artificial Sequence
  Synthetic oligonucleotide
caaggtaggtcttgaactcc

SEQ ID NO: 2 - DNA, 22 bp, Artificial Sequence
  Synthetic oligonucleotide
agctctggtgaaggtctttctc

SEQ ID NO: 3 - DNA, 26 bp, Artificial Sequence
  Synthetic oligonucleotide
acagttgtgagccaccgtacccggcc

SEQ ID NO: 4 - DNA, 15 bp, Artificial Sequence
  Synthetic oligonucleotide
cacgtgcgtgcaggt

SEQ ID NO: 5 - DNA, 7 bp, Artificial Sequence
  Synthetic oligonucleotide
tacgtca

SEQ ID NO: 6 - DNA, 15 bp, Artificial Sequence
  Synthetic oligonucleotide
cacgtgcgtgcgggt

SEQ ID NO: 7 - DNA, 7 bp, Artificial Sequence
  Synthetic oligonucleotide
tacatca

SEQ ID NO: 8 - DNA, 25 bp, Artificial Sequence
  Synthetic oligonucleotide
acaggcgtgagcactgctcctggcc

SEQ ID NO: 9 - DNA, 70 bp, Homo sapiens
tagagatggtgtttcacaatgttggcaaggtaggtcttgaactcctgacctcaagtgatctgcccgcctc

SEQ ID NO: 10 - DNA, 20 bp, Artificial Sequence
  Synthetic oligonucleotide
ggcaaggtaggtcttgaact

SEQ ID NO: 11 - DNA, 20 bp, Artificial Sequence
  Synthetic oligonucleotide
ttggcaaggtaggtcttgaa

SEQ ID NO: 12 - DNA, 20 bp, Artificial Sequence
  Synthetic oligonucleotide
tgttggcaaggtaggtcttg

SEQ ID NO: 13 - DNA, 20 bp, Artificial Sequence
  Synthetic oligonucleotide
aatgttggcaaggtaggtct

SEQ ID NO: 14 - DNA, 20 bp, Artificial Sequence
  Synthetic oligonucleotide
acaatgttggcaaggtaggt

SEQ ID NO: 15 - DNA, 20 bp, Artificial Sequence
  Synthetic oligonucleotide
aggtaggtcttgaactcctg

SEQ ID NO: 16 - DNA, 20 bp, Artificial Sequence
  Synthetic oligonucleotide
gtaggtcttgaactcctgac

SEQ ID NO: 17 - DNA, 20 bp, Artificial Sequence
  Synthetic oligonucleotide
aggtcttgaactcctgacct

SEQ ID NO: 18 - DNA, 20 bp, Artificial Sequence
  Synthetic oligonucleotide
gtcttgaactcctgacctca

SEQ ID NO: 19 - DNA, 20 bp, Artificial Sequence
  Synthetic oligonucleotide
cttgaactcctgacctcaag

SEQ ID NO: 20 - DNA, 22 bp, Artificial Sequence
  Synthetic oligonucleotide
ggcaaggtaggtcttgaactcc

SEQ ID NO: 21 - DNA, 24 bp, Artificial Sequence
  Synthetic oligonucleotide
ttggcaaggtaggtcttgaactcc

SEQ ID NO: 22 - DNA, 26 bp, Artificial Sequence
  Synthetic oligonucleotide
tgttggcaaggtaggtcttgaactcc

SEQ ID NO: 23 - DNA, 28 bp, Artificial Sequence
  Synthetic oligonucleotide
aatgttggcaaggtaggtcttgaactcc

SEQ ID NO: 24 - DNA, 22 bp, Artificial Sequence
  Synthetic oligonucleotide
caaggtaggtcttgaactcctg

SEQ ID NO: 25 - DNA, 24 bp, Artificial Sequence
  Synthetic oligonucleotide
caaggtaggtcttgaactcctgac

SEQ ID NO: 26 - DNA, 26 bp, Artificial Sequence
  Synthetic oligonucleotide
caaggtaggtcttgaactcctgacct

SEQ ID NO: 27 - DNA, 28 bp, Artificial Sequence
  Synthetic oligonucleotide
caaggtaggtcttgaactcctgacctca

SEQ ID NO: 28 - DNA, 24 bp, Artificial Sequence
  Synthetic oligonucleotide
ggcaaggtaggtcttgaactcctg

SEQ ID NO: 29 - DNA, 28 bp, Artificial Sequence
  Synthetic oligonucleotide
ttggcaaggtaggtcttgaactcctgac

SEQ ID NO: 30 - DNA, 32 bp, Artificial Sequence
  Synthetic oligonucleotide
tgttggcaaggtaggtcttgaactcctgacct

SEQ ID NO: 31 - DNA, 36 bp, Artificial Sequence
  Synthetic oligonucleotide
aatgttggcaaggtaggtcttgaactcctgacctca

(19) United States
(12) Patent Application Publication
(10) Pub. No.: US 2006/0269925 A1
(43) Pub. Date: Nov. 30, 2006

(54) Methods for determining glutathione S-transferase theta-1 genotype

(75) Inventors: Fabio Nunes, Boston, MA (US); Mia MacCollin, Medford, MA (US); Iris Ahronowitz, Medford, MA (US)

Correspondence Address: WOLF GREENFIELD & SACKS, PC, FEDERAL RESERVE PLAZA, 600 ATLANTIC AVENUE, BOSTON, MA, 02210-2206, US

(73) Assignee: The General Hospital Corporation, Boston, MA (US)

(21) Appl. No.: 11/137,696

(22) Filed: May 25, 2005

(51) Int. Cl. C12Q 1/68; C12P 19/34

(52) U.S. Cl. 435/6; 435/91.2

(57) ABSTRACT

The invention relates to methods for determining GSTT1genotype, and the diagnostic and prognostic uses of these methods.

### GOVERNMENT INTEREST

This work was funded in part by the National Institutes of Health under grant number R01 NS40527-01A2. The government may have certain rights in this invention.

### FIELD OF THE INVENTION

The invention relates to methods for determining GSTT1 genotype, and the diagnostic and prognostic uses of these methods.

### BACKGROUND OF THE INVENTION

Meningiomas are among the most common human brain tumors, accounting for 15-26% of all intracranial neoplasms, with an incidence in the general population of 6-7.8 per 100,000 individuals (DeAngelis, L. M. (2001) N Engl J Med 344(2): 114-23; Whittle, I. R., C. Smith, et al. (2004) Lancet 363(9420): 1535-43). Although meningiomas can affect people of all ages, they present primarily between the fourth and sixth decades of life, with an increased incidence among women. Clinical features related to meningiomas usually depend on the site of origin and are caused by compression or reactive changes of the adjacent parenchyma. Meningiomas are composed of neoplastic arachnoidal cells and may progress to atypical and malignant tumors (Jaaskelainen, J., M. Haltia, et al. (1986) Surg Neurol 25(3): 233-42; Perry, Jenkins et al. 1996; Leuraud, P., E. Dezamis, et al. (2004) J Neurosurg 100(2): 303-9).

Treatment of meningiomas is primarily surgical, with complete resection of the tumors located in accessible sites. However, tumors involving vital neural or vascular structures, tumors en plaque, and higher grade lesions frequently require partial resection followed by radiation therapy (Samii, M., G. A. Carvalho, et al. (1996) J Neurosurg 84(3): 375-81; Nakamura, M. and M. Samii (2003) ActaNeurochir (Wien) 145(3): 215-9, discussion 219-20). Although so much has been studied about meningiomas in the past century, to date there are frustratingly few therapeutic options for tumors that cannot be completely resected.

Neurofibromatosis 2 (NF2) is an autosomal dominant disease affecting 1 in every 40,000 individuals. The hallmark of NF2 is the occurrence of bilateral vestibular schwannomas, but other intracranial and spinal tumors can also occur. Meningiomas occur in half of the NF2 patients and are frequently a great cause of morbidity and mortality with few therapeutic options (Evans, D. G., S. M. Huson, et al. (1992) Q J Med 84(304): 603-18; Parry, D. M., R. Eldridge, et al. (1994) Am J Med Genet 52(4): 450-61; Mautner, V. F., M. Lindenau, et al. (1996) Neurosurgery 38(5): 880-5 discussion 885-6). Treatment of NF2-associated tumors is also primarily surgical, but occurrence of multiple tumors makes it impossible to operate on all of them.

Studies of genotype-phenotype relationships in NF2 have clearly defined a relationship between overall severity (measured by age of onset and deafness) and NF2 mutation type, phenotypic variables which run true in families (Evans, D. G., L. Trueman, et al. (1998) J Med Genet 35(6): 450-5; Nunes, F. and M. MacCollin (2003) J Child Neurol 18(10): 718-24; Baser, M. E., L. Kuramoto, et al. (2004) Am J Hum Genet 75(2): 231-9). Conversely, both the presence of meningiomas and the meningioma tumor load frequently varies between affected family members, and the genetic or epigenetic basis for this important variability has not yet been established.

The neurofibromatosis 2 gene (NF2) on chromosome 22 is the initiating event in 50-60% of sporadic meningiomas. Moreover, alterations in the GSTT1 (Glutathione-S-Transferase Theta 1) transcript, also on chromosome 22, have recently been associated with an increased risk of symptomatic meningioma development.

Glutathione S-transferase theta-1 (GSTT1) is an enzyme that detoxifies certain environmental toxins. Reduced GSTT1 has been correlated with increased risk or incidence of a number of cancers. GSTT1 can be assayed by examining enzyme activity and by genotyping. As a result of genotyping, it is known that the population has individuals that are homozygous for active GSTT1, that are homozygous for inactivated (deletion) GSTT1, and heterozygotes that have one of each type of GSTT1 allele.

Based on the risk of cancer in persons having reduced or zero GSTT1 activity, there is interest in screening persons for their GSTT1 genotypes. Two types of PCR-based genotyping assays have been developed. The first type identifies only the presence or absence of the active GSTT1 allele using a single PCR reaction, but does not determine which individuals are heterozygous, and does not control for PCR reaction failure. The second type uses two PCR reactions, enabling identification of heterozygotes, but also does not control for PCR reaction failure and is more cumbersome than the first type of test.

### SUMMARY OF THE INVENTION

We have developed a GSTT1 genotyping method that uses a single PCR reaction, but which can identify GSTT1 heterozygotes and controls for PCR reaction failure. This method permits identification of all three genotypes and avoids the lack of certainty inherent in the existing GSTT1 genotyping assays.

The GSTT1 assays of the invention permit development of new prognostic tests and staging tests for meningioma and any other disorders or conditions in which a reduction of GSTT1 activity causes or worsens the disorder or condition. In addition, identification of a defined subset of meningioma patients most likely to progress makes it possible to target them for early adjunct therapy. Further, identification of patients having deleted GSTT1 permits treatment of such patients to replace GSTT1 or augment other GST enzyme expression. Similarly, genetic counseling and lifestyle changes could be recommended for patients at risk for meningioma initiation.

The new PCR-based GSTT1 genotyping method is based on the gene structure of GSTT1. Recombination between the repetitive elements HA5 and HA3 that flank the GSTT1 gene leads to deletion of a portion of the GSTT1 locus. We have designed primers that anneal at the 3′ ends of both HA5 and HA3 in accordance with the sequence similarity of these elements. For an intact GSTT1 allele, amplification occurs with 3′ primer annealed to the 3′ end of HA5, because the HA3 element primer binding site is located 54 kb away. For a deleted GSTT1 allele, amplification occurs with the 3′ primer annealed to the HA3 element, because the 3′ end of the HA5 element is deleted. A common 5′ primer anneals to the 5′ end of the HA5 element that is present in both intact and deleted GSTT1 alleles. Both amplification reactions yield an amplification product of the same size (e.g., 709 bp using the exemplary primer used in the Examples). A lack of an amplification product indicates failure of the PCR reaction.

In some embodiments, the two amplification products are differentiated by cleavage with a restriction enzyme, e.g., using HpyCH4IV to cleave at ACGT sites as shown in the Examples. The amplification product of the deleted GSTT1 allele has a single restriction site, generating two fragments of 483 bp and 221 bp. The amplification product of the active GSTT1 allele has an additional restriction site, yielding three fragments of 483 bp, 127 bp and 94 bp. Amplification of DNA from a GSTT1 heterozygote yields all four fragments. These fragments are readily resolved, e.g., on an acrylamide gel.

Thus, according to one aspect of the invention, methods for determining the genotype of GSTT1 in a subject are provided. The methods include amplifying fragment(s) of GSTT1 by subjecting a genomic DNA sample of the subject to DNA amplification using a 5′ primer and a 3′ primer that together amplify fragments of intact GSTT1 alleles and/or deleted GSTT1 alleles. The 5′ primer anneals to a first HA5 element sequence or to a DNA region located immediately adjacent and upstream (5′) of HA5, the 3′ primer anneals to a second HA5 element sequence and to a HA3 element sequence, and each of the second HA5 element sequence and the HA3 element sequence are spaced apart from and 3′ to the first HA5 element sequence.

In some embodiments, a fragment amplified by the 5′ primer and the 3′ primer annealed to the second HA5 element sequence is about the same length as a fragment amplified by the 5′ primer and the 3′ primer annealed to the HA3 element sequence.

In other embodiments, the methods further include digesting the amplified fragment(s) with a restriction enzyme that differentially cleaves the amplified fragment(s) depending on whether the 3′ primer annealed to the second HA5 element sequence or to the HA3 element sequence.

In additional embodiments, the size of the fragment(s) is determined by gel electrophoresis.

In certain preferred embodiments, the genomic DNA sample of the subject is obtained from blood or from a tumor.

The 5′ primer preferably comprises SEQ ID NO:1, and more preferably consists of SEQ ID NO:1. The 3′ primer preferably comprises SEQ ID NO:2 and more preferably consists of SEQ ID NO:2. In some preferred embodiments, the 5′ primer is located up to about 1 kb in the 5′ direction from the HA5 element. Preferably the 5′ primer is located up to about 1 kb in the 5′ direction from SEQ ID NO:1.

In preferred embodiments, the DNA amplification used in the genotyping methods is polymerase chain reaction (PCR).

According to another aspect of the invention, kits for genotyping GSTT1 are provided. The kits include a first container containing a 5′ primer, and a second container containing a 3′ primer. The 5′ primer anneals to a first HA5 element sequence. The 3′ primer anneals to a second HA5 element sequence and/or to a HA3 element sequence; each of the second HA5 element sequence and the HA3 element sequence are spaced apart from and 3′ to the first HA5 element sequence. The 5′ primer and the 3′ primer together amplify fragments of intact GSTT 1 alleles (if the second primer anneals to the second HA5 element sequence) and/or deleted GSTT1 alleles (if the second primer anneals to the HA3 element sequence) in a DNA amplification reaction.

In preferred embodiments, the 5′ primer is SEQ ID NO:1, and/or the 3′ primer is SEQ ID NO:2.

The kits in other embodiments also include a third container containing a DNA restriction enzyme that differentially cleaves the amplified fragment(s) depending on whether the 3′ primer annealed to the second HA5 element sequence or to the HA3 element sequence. The kits also can include one or more containers containing buffer solution(s), DNA polymerase enzyme(s), restriction enzyme(s) and/or nucleotide solution(s). The DNA polymerase enzyme preferably is a thermostable DNA polymerase.

In still other embodiments, the kits also include one or more containers containing GSTT1 control DNA. In these embodiments, it is preferred that the GSTT1 DNA is homozygous for GSTT1 wild type alleles, homozygous for GSTT1 null alleles and/or heterozygous for GSTT1 wild type and null alleles.

According to a further aspect of the invention, methods for determining tumor growth and progression in a subject are provided. The methods include obtaining a genomic DNA sample from the subject, and determining the genotype of GSTT1 in the genomic DNA sample according to any of the foregoing methods. A GSTT1 null genotype indicates that the subject has or will have elevated tumor growth and progression.

In some embodiments, the tumor is one or more meningiomas, or is one or more bladder cancers, squamous cell carcinomas, cancers in the upper aero digestive tract, gastric cancers, acute lymphoblastic leukaemias, hepatocellular carcinomas, cervical cancers, breast cancers, lung cancers, acute myeloid leukemias, thyroid cancers, astrocytomas, prostate cancers, hepatocellular carcinomas, colon cancers, bladder cancers or chronic lymphoblastic leukemias.

In other embodiments, the subject has or is suspected of having neurofibromatosis 2 (NF2).

According to yet another aspect of the invention, methods for determining prognosis of a subject are provided. The methods include determining a GSTT1 genotype of the subject according to any of the foregoing methods, wherein a homozygous null GSTT1 genotype or a heterozygous GSTT1 genotype is indicative of a relatively poor prognosis for the subject, and wherein a homozygous wild type GSTT1 genotype is indicative of a relatively good prognosis for the subject.

In some embodiments, the subject has or is suspected of having one or more meningiomas. In other embodiments, the subject has or is suspected of having one or more bladder cancers, squamous cell carcinomas, cancers in the upper aero digestive tract, gastric cancers, acute lymphoblastic leukaemias, hepatocellular carcinomas, cervical cancers, breast cancers, lung cancers, acute myeloid leukemias, thyroid cancers, astrocytomas, prostate cancers, hepatocellular carcinomas, colon cancers, bladder cancers or chronic lymphoblastic leukemias.

In still other embodiments, the subject has or is suspected of having neurofibromatosis 2 (NF2).

According to a further aspect of the invention, methods are provided for determining the suitability of therapeutic intervention for a patient having or suspected of having one or more meningiomas. The methods include determining a GSTT1 genotype of the subject according to any of the foregoing methods. A homozygous null GSTT1 genotype indicates that therapeutic intervention is suitable.

In some embodiments, the therapeutic intervention is GST replacement therapy, preferably including administration of an effective amount of GSTT1 to the patient. In other embodiments, the therapeutic intervention is surgery.

In certain embodiments, the meningioma patients are NF2 patients, e.g., intracranial meningiomas.

These and other objects and embodiments of the invention will be described in further detail in connection with the detailed description of the invention.

### BRIEF DESCRIPTION OF THE DRAWINGS

FIG. 1 shows a schematic depiction of GSTT1 deletion. GSTT1 is located on 22q11.2 centromeric to the NF2 gene. Recombination between HA5 and HA3, two repetitive elements more than 90% identical, leads to deletion of the entire GSTT1 gene. Genotype-phenotype studies have shown a gene-dose effect, with individuals heterozygous for the GSTT1 deletion showing decreased enzyme activity compared to individuals homozygous for the active allele (Warholm, et al., 1995, Sprenger et al., 2000). Individuals homozygous for the GSTT1 deleted allele have no enzyme activity.

FIG. 2 shows GSTT1 deletion breakpoints and the PCR assay. Recombination between two repetitive elements flanking GSTT1 (HA3 and HA5) leads to deletion of the entire GSTT1 gene and formation of a recombinant repetitive element (HA0). Genomic sequences of HA5 and HA3 as well as the sequence of the recombinant segment (HA0) are shown above. HA0 is composed of the 5′ of HA5 followed by a 403 basepairs fragment 100% identical to HA5 and HA3, continuous with the 3′ of HA3. Basepair differences between HA5 and HA3 are marked with a # sign. Our PCR assay takes advantage of the sequence similarities and individual basepair differences between HA5 and HA3. By designing our reverse primer (P2) to anneal to an area of sequence similarity between HA5 and HA3 we were able to amplify the active allele (dashed line) and the deleted allele (solid line) using the same PCR reaction. PCR amplification is followed by restriction enzyme digestion of a restriction site present in both alleles (constant restriction site serves as a control for digestion). A second restriction site present only in the active allele is used to differentiate the alleles. As a result, the GSTT1 is digested in only two PCR fragments (483+221 basepairs), while an additional restriction site present only in the active allele results in three detectable PCR fragments (483+127+94 basepairs). The graphic representation is adapted from Sprenger et al., 2000. Sequences shown in HA5 (left to right): SEQ ID NO:3 (acagttgtgagccaccgtacccggcc), SEQ ID NO:4 (cacgtgcgtgcaggt) and SEQ ID NO:5 (tacgtca). Sequences shown in HA0 (left to right): SEQ ID NO:3, SEQ ID NO:6 (cacgtgcgtgcgggt) and SEQ ID NO:7 (tacatca). Sequences shown in HA3 (left to right): SEQ ID NO:8 (acaggcgtgagcactgctcctggcc), SEQ ID NO:6 and SEQ ID NO:7.

FIG. 3 shows the results of the GSTT1 PCR-based assay. Samples can be rapidly genotyped by visualizing the restriction fragments on an 8% polyacrylamide gel. PCR amplification of both alleles yielded a 709 basepair (bp) product. After digestion with HpyCH4IV, all samples had a 483 bp fragment due to digestion of the constant restriction site. In addition to this constant band, a homozygous positive individual (sample 1) had two more detectable fragments (127 bp and 94 bp), while a homozygous null individual (sample 2) had a 483 bp and a 221 bp band. Heterozygous individuals (sample 3) exhibited all four restriction fragments (483 bp, 221 bp, 127 bp and 94 bp).

FIG. 4 shows that loss of heterozygosity (LOH) of 22q leads to decreased GSTT1 activity in the tumor tissue. Monosomy or terminal deletion of chromosome 22 caused by NF2 LOH leads to loss of the GSTT1 allele located cis to the NF2 wildtype allele lost. In patients homozygous null for GSTT1, LOH will have no influence in the enzyme activity present in tumor tissue. However, patients homozygous for the active GSTT1 will always lose an active GSTT1 allele during the LOH process, showing decreased enzyme activity in tumor tissue. Patients heterozygous for GSTT1 will lose either the deleted GSTT1 allele retaining the active allele in the tumor, or will lose the active GSTT1 allele with no enzyme activity remaining in the tumor tissue. Gray circle or minus sign=deleted GSTT1 allele; black circle or plus sign=active GSTT1 allele.

FIG. 5 shows LOH of 22q leading to loss of active GSTT1 allele. Monosomy or terminal deletion of chromosome 22 in sporadic meningiomas due to 22q LOH will lead to loss of the wild-type NF2 allele, as well as loss of the GSTT1 allele located cis to the NF2 allele lost. Polyacrylamide gel of microsatellite marker CryBB2, located centromeric to NF2, shoes loss of the top band in the tumor compared to the blood sample (*). When analyzed for GSTT1 status in the blood and tumor this patient was found to be heterozygous for GSTT1 in genomic DNA, but 22q LOH led to loss of the remaining GSTT1 active allele in the tumor.

### DETAILED DESCRIPTION OF THE INVENTION

We propose that GSTT1 activity is a fundamental factor in determining individual susceptibility to meningioma initiation and subsequent progression. We base our proposal on the observations that 1) GSTT1 activity is determined by a polymorphism resulting in gene deletion, with the null allele present in 20% of the general population, 2) GSTT1 null allele has been associated with increased risk of meningioma initiation (resection) and 3) monosomy of chromosome 22 is the main mechanism of neurofibromatosis 2 gene (NF2) loss of heterozygosity (LOH) in meningiomas, leading to loss of the GSTT1 allele located cis to the NF2 wild type allele. Our preliminary studies corroborate previous reports showing that LOH of 22q is correlated with tumor progression. Moreover, preliminary experiments have also shown that patients carrying homozygous deletion of GSTT1 in genomic DNA, and patients heterozygous for GSTT1 that develop LOH of 22q with consequent loss of the active allele of GSTT1, tend to progress to higher grades of meningioma.

Glutathione S-Transferase (GST) is a large family of genes involved in the metabolism of many xenobiotics, including an array of environmental carcinogens and chemotherapeutic agents. There are at least seven different families of GSTs reported to date: alpha (α), mu (μ), pi (π), sigma (σ), theta (θ), kappa (κ), and zeta (ξ) (Landi 2000). This classification is based on substrate specificity, chemical affinity, protein structure, amino-acid sequence, and kinetic behavior. GSTT1 (GenBank accession number AF240786) is an isoenzyme involved in the cellular detoxification system known to catalyze the conjugation of glutathione with different species of electrophilic compounds including dichloromethane, ethylene-dibromide, ethylene-oxide, and a number of other potentially carcinogenic halogenated compounds (Pemble, Schroeder et al. 1994). Interestingly, the GSTT1 activity in humans is polymorphic, with a non-conjugator phenotype first discovered by lack of glutathione conjugation in human erythrocytes (Schroder, Hallier et al. 1992). Using incubation of methyl bromide and gas chromatography in samples of human erythrocytes the conjugator phenotype was initially found in 75% of the individuals, while non-conjugator phenotype, defined as no change in the gas concentration of methyl-bromide using the head space technique, was reported in the remaining 25% (Hallier, Langhof et al. 1993). Interestingly, in humans, glutathione-dependent conjugation by GSTT1 is polymorphic, with a full-conjugator, a partial-conjugator, and a non-conjugator phenotype, as measured by their ability to conjugate methyl chloride in their erythrocytes (Pemble, Schroeder et al. 1994; Warholm, Rane et al. 1995). Even before the gene could be identified, associations between the non-conjugator phenotype and induction of sister chromatid exchanges in lymphocytes had already been suggested (Hallier, Langhof et al. 1993).

The technique used to genotype GSTT1 in almost all of these studies uses a set of primers to amplify a fragment of the gene by polymerase chain reaction (PCR). In the absence of a detectable PCR product the individuals are classified as GSTT1 null genotype, or non-conjugators, while cases in which a PCR product is detected the individuals are classified as GSTT1 positive genotype, or conjugators. Using this genotyping method it is impossible to differentiate between full-conjugators and partial-conjugators. This method also has the disadvantage of not reliably controlling for PCR failures since the absence of PCR product among non-conjugators is only controlled by amplification of a different PCR product (β-globin gene) located outside of the GSTT1 gene-deletion segment, which amplifies independently of GSTT1. More specifically, one study of 1277 individuals with brain tumors reported that up to 9% of the samples studied failed to amplify for GSTT1, possibly confounding the GSTT1 genotyping results (De Roos, Rothman et al. 2003).

To solve this problem we have developed a new PCR based assay in which all three GSTT1 genotypes can be clearly identified and at the same time reliably controls for PCR failures. Recently, a new genotyping method was proposed which can differentiate between full-conjugators and partial conjugators (Sprenger, Schlagenhaufer et al. 2000). The Sprenger method uses two different PCR fragments to identify the active and deleted GSTT1 alleles. Although th Sprenger method allows identification of all three possible genotypes by PCR analysis, it is also susceptible to amplification failures. The newly developed PCR assay disclosed herein does not require two separate amplification reactions, and internally controls for amplification failure.

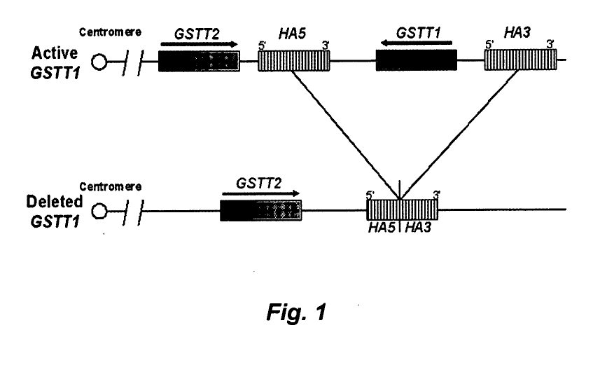

The GSTT1 gene, located on 22q11.2, was cloned in 1994 (Pemble, Schroeder et al. 1994). Shortly after the cloning of the gene the molecular genetic basis of the biochemical polymorphism in humans was revealed to be a deletion of the entire GSTT1 gene due to a recombination event between HA5 and HA3 (FIG. 1). Individuals homozygous for the deleted allele have a non-conjugator phenotype, while presence of one or two active GSTT1 alleles have been shown by enzymatic assays to confer a partial- or full-conjugator phenotype, respectively. The frequency of the null genotype varies significantly among different populations. For example, 64% of the Chinese population but only 10-25% of Caucasians carry the null genotype (Nelson, Wiencke et al. 1995; Warholm, Rane et al. 1995; Jourenkova-Mironova, Voho et al. 1999). In the US the GSTT1 null genotype was reported in 20% of Caucasians and 24% of African-Americans, but some regional differences are seen, with a null genotype present in only 15% of Caucasians in the New England region (Nelson, Wiencke et al. 1995).

The relationship between GSTT1 null genotype and initiation of a number of cancers occurring in several human tissues including bladder, colon, breast, and brain has been reported (Table 1). Conflicting results have been published regarding GSTT1 null genotype and increased incidence of brain tumors. Initial reports of glioma initiation showed a frequency of GSST1 null genotype in 32% of cases, compared to 18% of controls (Elexpuru-Camiruaga, Buxton et al. 1995), but successive reports failed to confirm this association (Ezer, Alonso et al. 2002; De Roos, Rothman et al. 2003; Wrensch, Kelsey et al. 2004). In the same study, the frequency of GSTT1 null genotype among patients with sporadic meningiomas was found to be even higher than among gliomas, present in 45% of the meningioma cases compared to 18% of controls (odds ratio=3.57, exact P=0.0002). In a second study, a somewhat weaker association was found between the GSTT1 null genotype and meningioma initiation (odds ratio=1.5), with, however, an interesting association between GSTT1 null genotype and meningioma initiation at a younger age (age <40 years, odds ratio=2. 1; age >60 years odds ratio=1.4) (De Roos, Rothman et al. 2003). This difference in age groups is consistent with our hypothesis that meningiomas become symptomatic (progress) earlier in life among individuals with no GSTT1 activity. Furthermore, these studies have used genotyping technology which groups GSTT1 heterozygotes with full conjugators (homozygous active) and compares them to GSTT1 null genotype (homozygous deleted). We hypothesize that heterozygotes may also have increased risk for meningioma progression and that with our ability to distinguish all three genotypes will come a clearer picture of the relative risk to patients with reduced GSTT1 activity. In addition, the ability to genotype GSTT1 effectively using the methods of the invention also provides diagnostic and prognostic methods for additional cancers in which reduced GSTT1 activity contributes to progression, such as bladder cancer, squamous cell carcinoma, cancers in the upper aero digestive tract, gastric cancer, acute lymphoblastic leukaemia, hepatocellular carcinoma, cervical cancer, breast cancer, lung cancer, acute myeloid leukemia, thyroid cancer, and the cancers listed in Table 1.

TABLE 1
GSTT1 null genotype and tumor initiation. Several studies have
reported a correlation between the presence of the GSTT1 null
genotype and an increased risk of tumor initiation or progression.
However, all of these studies have been unable to determine the
difference between patients carrying one or two active GSTT1
alleles. To date, no study has looked at the risk of tumor initiation
associated with the heterozygous genotype of GSTT1. Note the
GSTT1 null genotype difference seen among control groups in the
different populations studied.

| | Risk conferred by the GSTT1 | |
|---|---|---|
| | null genotype or frequency | |
| | of the null genotype among | |
| Tumor type | patients and controls | Reference |
| Meningioma | OR 1.5 to 4.52 | (Elexpuru-Camiruaga, |
| | | Buxton et al. 1995; De |
| | | Roos, Rothman et al. |
| | | 2003) |
| Astrocytoma | OR 2.67 | (Elexpuru-Camiruaga, |
| | | Buxton et al. 1995) |
| Prostate | OR 1.8 | (Srivastava, Mandhani |
| cancer | | et al. 2005) |
| Hepatocellular | Null genotype among patients | (Deng, Wei et al. 2005) |
| carcinoma | 59% | |
| | Null genotype among controls | |
| | 42% | |
| Colon cancer | OR 1.42 | (Chen, Jiang et |
| | | al. 2005) |
| Bladder cancer | OR 2.0 to 4.93 | (Brockmoller, Cascorbi |
| | | et al. 1996; Abdel- |
| | | Rahman, Anwar et al. |
| | | 1998; Sobti, Al-Badran |
| | | et al. 2005) |
| Chronic | Null genotype among patients | (Tsabouri, Georgiou et |
| Lymphoblastic | 74% | al. 2004) |
| leukemia | Null genotype among controls | |
| | 36% | |

The Molecular Biology of Sporadic Meningioma Initiation

The molecular origin of meningiomas has not yet been fully explained. Loss of heterozygosity (LOH) of chromosome 22, including the NF2 region at 22q12, occurs in approximately half of all sporadic meningiomas (Ruttledge, Sarrazin et al. 1994; Leuraud, 15 Marie et al. 2000). The genetic basis of sporadic meningiomas not inactivated at the NF2 locus remains unclear. Losses involving the short arm of chromosome one have been described in approximately 30% of sporadic meningiomas, but were associated with inactivation of NF2 in the majority of the tumors (Leone, Bello et al. 1999; Leuraud, Marie et al. 2000). Comparative genomic hybridization (CGH) has also been used to examine the gains and losses of genetic material occurring in meningiomas. Loss of chromosomes 22 (in 50% of tumors) and 1p (in 33% of tumors) are also the main molecular events described in tumors studied by this technique (Khan, Parsa et al. 1998; Ozaki, Nishizaki et al. 1999; Arslantas, Artan et al. 2002; Arslantas, Artan et al. 2003). Other chromosome losses reported include 4q, 6q, 8q, 9p, 10q, 13q, 14q, 15q, 17p, 18q, 19p, X, and Y, as well as gains of 12q, 15q, and 18p (Khan, Parsa et al. 1998; Arslantas, Artan et al. 2002; Arslantas, Artan et al. 2003). Although several chromosome arms have been implicated in meningioma tumorigenesis by CGH and LOH analysis, only a few genes have been individually analyzed, including DAL-1, p18, TP53, PTEN, KRAS, NRAS, HRAS, CDKN2A, P14ARF, CDKN2B and CDKN2C (Gutmann, Donahoe et al. 2000; Leuraud, Marie et al. 2000; Bostrom, Meyer-Puttlitz et al. 2001; Joachim, Ram et al. 2001).

Mechanisms of LOH formation vary between tumor types and may occur by physical loss of an entire chromosome or part of a chromosome (including monosomy, terminal deletion, and interstitial deletion), or by duplication of the mutated allele (as seen in mitotic recombination or mitotic non-disjunction). Recent reports, including studies by the PI (see also preliminary data), shows that in sporadic meningiomas, LOH of the NF2 gene occurs by monosomy or terminal deletion of almost the entire long arm of chromosome 22 (Ozaki, Nishizaki et al. 1999; Arslantas, Artan et al. 2002; Arslantas, Artan et al. 2003). These findings suggest that one copy of every gene on chromosome 22, and not only NF2 itself, is lost in the process. It can also be predicted that the GSTT1 allele located cis to the wild-type NF2 allele will be lost in this process. If the GSTT1 allele lost is an active allele, GSTT1 enzyme activity in the tumor tissue will be decreased by half or completely absent, depending on the patient's original genotype. To date, no study has looked at the effect of additional loss of GSTT1 activity in tumor due to 22q loss.

The Molecular Biology of Sporadic Meningioma Progression

Malignant progression of benign tumors has been well documented in different tumor types with the theory of clonal evolution being widely accepted. In this theory, benign tumors develop more molecular alterations as they progress to atypical and malignant grade. However, meningioma progression has been shown to follow a different pathway, in which all molecular alterations seen in high-grade tumors were shown to be present in benign tumors even before tumor progression (Al-Mefty, Kadri et al. 2004; Leuraud, Dezamis et al. 2004). More specifically, one study in which histological progression could be confirmed in tumor recurrences, genetic analysis of 11 samples from four different patients showed that the presence of a complex karyotype in the benign tumor preceded the histopathologically confirmed progression (Al-Mefty, Kadri et al. 2004). Among the alterations related to meningioma progression, loss of chromosome 22 is the most commonly seen, present in 33-48% of benign meningiomas (Perry, Jenkins et al. 1996; Ozaki, Nishizaki et al. 1999; Leuraud, Dezamis et al. 2004), 68-87% of atypical tumors (Leone, Bello et al. 1999; Ozaki, Nishizaki et al. 1999; Leuraud, Dezamis et al. 2004), and almost 100% of malignant meningiomas (Leone, Bello et al. 1999; Ozaki, Nishizaki et al. 1999; Leuraud, Dezamis et al. 2004). Other frequent allelic losses seen in meningioma progression occur in chromosome 1p, 10q, and 14q (Leone, Bello et al. 1999; Ozaki, Nishizaki et al. 1999; Al-Mefty, Kadri et al. 2004; Leuraud, Dezamis et al. 2004). The fact that 22q LOH is present as an early event in benign tumors but also has an increased frequency among malignant tumors corroborates the hypothesis that progression 22q LOH has a dual role in the molecular biology of meningiomas, and is involved in tumor initiation and progression. However, it is not clear why 22q LOH is present in almost 60% of the tumors, but only a small minority progresses to higher grades. We hypothesize that GSTT1 genotype in the patient's blood and tumor can influence tumor differentiation and progression. Therefore, the genotyping methods disclosed herein are useful in diagnosing and determining or predicting progression of tumors.

GSTT1 Genotyping Methods

The invention includes novel PCR-based assays of GSTT1 genotype and the use of such assays in diagnostic and prognostic methods, e.g., for analysis of initiation and progression of meningioma and other cancers.

The PCR-based GSTT1 genotyping method takes advantage of the gene structure of GSTT1. The method uses two primers to amplify a portion of the GSTT1 gene. Recombination between the repetitive elements HA5 and HA3 that flank the GSTT1 gene leads to deletion of a portion of the GSTT1 locus. A common 5′ primer anneals to the 5′ end of the HA5 element that is present in both intact and deleted GSTT1 alleles, or to a DNA region located immediately upstream (5′) of HA5. The 5′ primer is also referred to herein as a “forward” primer. The 3′ primer anneals at different locations depending on whether the recombination has occurred. Because there is sufficient sequence similarity between the 3′ end of the HA5 and HA3 elements, the 3′ primer is designed to anneal to either element. The 3′ primer is also referred to herein as a “reverse” primer.

The deletion event at the GSTT1 locus results in the loss of an approximately 54kb segment of the locus, which lost segment includes the 3′ end of the HA5 element. Thus, for amplification of an intact GSTT1 allele, the 3′ primer anneals to the 3′ end of HA5. The 3′ primer may also anneal to the HA3 element, but amplification from primers annealed to the HA3 element does not occur because the HA3 element primer binding site is located 54kb away. For amplification of a deleted GSTT1 allele, the 3′ primer anneals to the HA3 element only, because the 3′ end of the HA5 element is deleted. The 5′ primer anneals to the 5′ end of HA5 or to sequences immediately upstream (5′) of HA5 in the GSTT1 gene. The locations of the 5′ primer and the 3′ primer are selected such that the primer pair provides efficient amplification of DNA and yields amplification products that can be distinguished, preferably by the size of the cleavage products resulting from restriction endonuclease cleavage. Both amplification reactions yield an amplification product of the same size (e.g., 709 bp using the exemplary primers used in the Examples).

Obtaining an amplification product of the same size from the amplification of either intact or deleted GSTT1 alleles means that the efficiency of amplification is substantially equal between these reactions. This eliminates one source of variability generally found in PCR assays. Another advantage of the PCR assays described herein is that the lack of an amplification product indicates failure of the PCR reaction. This provides an important control for the success of the amplification reaction and eliminates one source of false results. A further advantage is that which is stated above: the assay uses a single pair of primers to amplify both intact and deleted alleles.

The specific primers exemplified herein are SEQ ID NO:1 (forward (5′) primer; caaggtaggtcttgaactcc) and SEQ ID NO:2 (reverse (3′) primer; agctctggtgaaggtctttctc). Additional primers are contemplated that amplify substantially the same portion of GSTT1, i.e., that anneal to HA5 and either the 3′ end of the HA5 or HA3 elements. For 5′ primers, preferred primers will be up to about 1 kb in the 5′ direction from the HA5 element, and more preferably up to about 1 kb in the 5′ direction from the most preferred primer location (SEQ ID NO:1) on the GSTT1 gene sequence. More preferably still, the 5′ primers are within about 500 nt, 400 nt, 300 nt, 250 nt, 200 nt, 150 nt, 100 nt, or 50 nt in the 5′ direction from SEQ ID NO:1. Suitable primer locations can be determined, for example, using the sequence represented by GenBank accession numbers AF240785 or AF240786.

The primers preferably are about 12, 13, 14, 15, 16, 17, 18, 19, 20, 21, 22, 23, 24, 25, 26, 27, 28, 29, 30, 31, 32, 33, 34, 35, 36 or more nucleotides in length and are non-overlapping to prevent formation of “primer-dimers”. Most preferably the primers are about 20-22 nucleotides in length. One of the primers hybridizes to one strand of the HA5 element (or 5′ thereto) of GSTT1 nucleic acid and the second primer hybridizes to the complementary strand of the 3′ end of the HA5 element of GSTT1 or the 3′ end of the HA3 element of GSTT1, in an arrangement which permits amplification of a portion of the GSTT1 nucleic acid. Selection of appropriate primer pairs is standard in the art.

Additional primers can be designed using available primer design software, or by extending the disclosed primers by adding nucleotides to the 5′ or 3′ ends of the primers. Alternatively, the size of primers can be kept constant (e.g., 20 nucleotides), and a different location of the primer(s) along the GSTT1 sequence can be selected, e.g., by conceptually “sliding” the primer toward the 5′ or 3′ ends of the GSTT1 sequence. Either or both primers can be altered in this manner; the altered primers can be tested in the manner disclosed herein to determine their effectiveness in amplification and GSTT1 genotyping.

For example, the following sequence (SEQ ID NO:9) represents the location of the 5′ primer (underlined) in the GSTT1 sequence, showing the 25 nucleotides that flank both ends of the primer sequence:
tagagatggtgtttcacaatgttggcaajgtaggtcttgaactcctgacctcaagtgatctgcccgcctc (SEQ ID NO:9).

Primers of 20 nucleotides can be readily selected from this sequence, as follows:

| SEQ | Primer sequence (portion |
|---|---|
| ID | of SEQ ID NO: 1 |
| NO | remaining is underlined) |
| 10 | ggcaaggtaggtcttgaact |
| 11 | ttggcaaggtaggtcttgaa |
| 12 | tgttggcaaggtaggtcttg |
| 13 | aatgttggcaaggtaggtct |
| 14 | acaatgttggcaaggtaggt |
| 15 | aggtaggtcttgaactcctg |
| 16 | gtaggtcttgaactcctgac |
| 17 | aggtcttgaactcctgacct |
| 18 | gtcttgaactcctgacctca |
| 19 | cttgaactcctgacctcaag |

These primers are exemplary of many similar primers that can be designed using the HA5 sequence and sequence upstream (5′) of HA5. One of ordinary skill in the art will recognize that primers containing other nucleotides can also be designed and prepared by routine experimentation.

Likewise, larger primers can be selected by adding nucleotides to the existing primer sequence:

| | |
|---|---|
| SEQ | Primer sequence (portion |
| ID | of SEQ ID NO: 1 |
| NO | remaining is underlined) |

Additions to 5′ end only:

| | |
|---|---|
| 20 | ggcaaggtaggtcttgaactcc |
| 21 | ttggcaaggtaggtcttgaactcc |
| 22 | tgttggcaaggtaggtcttgaactcc |
| 23 | aatgttggcaaggtaggtcttgaactcc |

Additions to 3′ end only:

| | |
|---|---|
| 24 | caaggtaggtcttgaactcctg |
| 25 | caaggtaggtcttgaactcctgac |
| 26 | caaggtaggtcttgaactcctgacct |
| 27 | caaggtaggtcttgaactcctgacctca |

Additions to 5′ and 3′ ends:

| | |
|---|---|
| 28 | ggcaaggtaggtcttgaactcctg |
| 29 | ttggcaaggtaggtcttgaactcctgac |
| 30 | tgttggcaaggtaggtcttgaactcctgacct |
| 31 | aatgttggcaaggtaggtcttgaactcctgacctca |

These sequences are exemplary of many similar primers that can be designed using the HA5 sequence and sequence upstream (5′) of HA5. Primers differing from SEQ ID NO:1 by additions of 2 nucleotides (for additions to the 5′ or 3′ ends) or by additions of 4 nucleotides (for additions to both ends) are shown. One of ordinary skill in the art will recognize that primers differing by other numbers of nucleotides can also be designed and prepared by routine experimentation.

The amplification products of intact and deleted GSTT1 alleles are conveniently distinguished by restriction digestion. The particular choice of restriction enzyme will depend on the amplification product that is analyzed, which in turn depends on the primers that are used to amplify the GSTT1 locus. For the preferred primers disclosed herein (SEQ ID NOs:1 and 2), the preferred restriction enzyme is HpyCH4IV, although HpyCH4V also will cleave the amplification products in a manner that will distinguish the amplification products of the null and normal GSTT1 alleles. If different primer sequence(s) are used for amplification, then one of ordinary skill in the art will understand that a different restriction enzyme can be selected in order to cleave the amplification product in a distinguishable manner.

The fragments of GSTT1 amplification products that are produced by restriction enzyme digestion are analyzed by any convenient means known to those of skill in the art. For example, gel electrophoresis (e.g., using agarose or acrylamide gels) can be used to distinguish the restriction fragments. Other methods include capillary electrophoresis, nucleic acid hybridization (e.g., microarrays having probes that distinguish the amplified products and restriction fragments) and mass spectrometry. Still other methods will be known to those skilled in the art.

The amplification using GSTT1-specific primers is carried out according to standard DNA amplification techniques. A preferred method of DNA amplification is polymerase chain reaction (PCR). An example of PCR amplification conditions using the preferred primers (SEQ ID NO:1 and SEQ ID NO:2) is provided in Example 1.

The methods of the invention are practiced on biological samples that contain DNA suitable for amplification. As used herein, a biological sample includes, but is not limited to: tissue (e.g., from a biopsy), cells, or body fluid (e.g. serum, blood, lymph node fluid, etc.). The fluid sample may include cells and/or fluid. The tissue and cells may be obtained from a subject or may be grown in culture (e.g. from a cell line).

The methods of determining the GSTT1 genotype may include use of labels to monitor the presence of the amplified molecules. Such labels may include, but are not limited to radiolabels or chemiluminescent labels, which may be utilized to determine whether and to what extent a GSTT1 nucleic acid molecule is amplified. For example, labeled nucleotides can be incorporated into DNA amplification methods, yielding labeled amplification products that can be detected using well known methods. As another example, labels can be used in the detection of amplification products, even though not incorporated into the amplification products themselves. For example, amplification products can be detected using labeled binding molecules, such as labeled antibodies, labeled hybridization probes, etc. In some cases the labeled molecules will be incorporated into molecules that specifically detect certain amplification products, in order to distinguish among amplification products. In other cases, labeled molecules will be incorporated into molecules that non-specifically label or detect the amplification products, with other means used to distinguish among the amplification products (e.g., size, length or molecular weight)

The invention also includes kits for genotyping GSTT1 by DNA amplification, including at least one pair of amplification primers which hybridize to GSTT1 and selectively amplify a portion of GSTT1. The kits typically contain separate containers containing the primers (although they may be mixed in a single container) and instructions for DNA amplification. The kits optionally include buffer solution(s), nucleotides, DNA polymerase (preferably thermostable, such as Taq DNA polymerase, Pfu DNA polymerase, PfuUltra™ hotstart DNA polymerase (Stratagene, La Jolla, Calif.), Vent_{R}® and DeepVent_{R}® polymerases (New England Biolabs, Beverly, Mass.)) and other components of DNA amplification reactions. The kits also may include components used for cleavage of the amplification products, such as restriction enzyme(s), buffer solution(s), etc. The kits further may include components of control reactions, such as samples of GSTT1 DNA to serve as a positive control, although as disclosed elsewhere herein, the assay design provides internal controls: for successful DNA amplification. Control components may include samples of genomic DNA that are homozygous for wild type GSTT1 alleles, homozygous for GSTT1 null alleles, or heterozygous.

The foregoing kits can include instructions or other printed material on how to use the various components of the kits for diagnostic purposes.

As used herein the term “control” means samples of materials tested in parallel with the experimental materials. Examples include samples from control populations, control samples generated through manufacture to be tested in parallel with the experimental samples, reagent controls, etc.

The invention further involves diagnostic and prognostic methods facilitated by determining the GSTT1 genotype of a subject. This determination is performed by assaying a biological sample from a subject for the GSTT1 genotype using the methods disclosed herein, i.e., amplification of a portion of GSTT1 DNA.

Thus, methods for determining tumor growth and/or progression in a subject are provided. The methods include obtaining a genomic DNA sample from the subject, and determining the genotype of GSTT1 in the genomic DNA sample according to any of the GSTT1 genotyping methods disclosed herein. As is demonstrated in the Examples below, GSTT1 null genotype indicates that NF2 patients have elevated tumor growth and progression. The method preferably is used for determining growth and/or progression of one or more meningiomas, although the method is applicable to other tumors in which a GSTT1 null genotype or a GSTT1 heterozygous genotype contributes to tumor growth and or progression. The subject of these methods preferably has or is suspected of having neurofibromatosis 2 (NF2).

The invention also includes methods for determining prognosis of a subject by determining a GSTT1 genotype of the subject according to the methods disclosed herein. A homozygous null GSTT1 genotype or a heterozygous GSTT1 genotype is indicative of a relatively poor prognosis, and a homozygous wild type GSTT1 genotype is indicative of a relatively good prognosis. The subject preferably will be one that has or is suspected of having one or more meningiomas, and can have or be suspected of having neurofibromatosis 2 (NF2).

The genotyping methods disclosed herein also can be use in determining the suitability of therapeutic intervention for a patient having or suspected of having one or more cancers, particularly meningiomas. A homozygous null GSTT1 genotype indicates that therapeutic intervention is suitable. Therapeutic intervention also may be suitable for patients having a heterozygous GSTT1 genotype. Possible therapeutic interventions include GST replacement therapy, e.g., administration of an effective amount of GSTT1 to the patient, and surgery. The method is particularly applicable to NF2 patients and other patients in which the meningiomas are intracranial meningiomas.

The methods disclosed herein also are applicable to tumors (or other disorders) in which a GSTT1 wild type homozygous genotype is implicated in initiation, growth and/or progression. As such, the methods are useful in a wide variety of clinical settings.

### EXAMPLES

### Example 1

### Development of a New Glutathione S-Transferase Theta-1 PCR-Based Assay.

Many previous reports have described a relation between GSTT1 null genotype and cancer. To date, however, there has been no straightforward way to determine heterozygous genotypes of GSTT1. We were interested in precisely determining the heterozygous status of GSTT1 because (a) we believe an increased risk of meningioma progression is found in patients heterozygous for GSTT1 compared to patients homozygous active and (b) because with NF2 loss of heterozygosity (LOH) in the tumor, an individual who was initially heterozygous for GSTT1 might lose the remaining active allele in the tumor tissue and increase even more their chances of tumor progression.

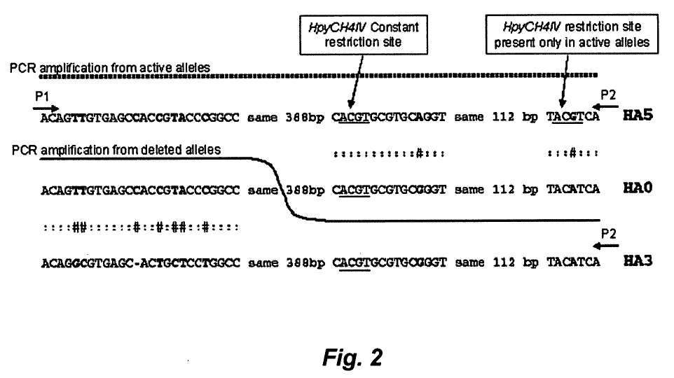

The deletion breakpoints of the GSTT1 polymorphism involve the recombination of two repetitive elements, HA5 and HA3, flanking the GSTT1 gene FIG. 2. After recombination between both repetitive elements, the GSTT1 deleted allele consists of the 5′ end of HA5 continuous with the 3′ end of HA3, deleting 54 kilobases (kb) including the entire GSTT1 gene (FIG. 1). Previous PCR-based assays allowed amplification from the active allele but not the deleted allele. Thus complete lack of amplification was inferred to be from the deleted allele while amplification would result from either a heterozygous or homozygous active and indeed these genotypes were indistinguishable.

Using carefully designed primers (see FIG. 2), we have been able to generate a PCR protocol in which PCR amplification occurs independently from either the deleted or the active GSTT1 allele. To accomplish this we have designed a forward primer (SEQ ID NO: 1: caaggtaggtcttgaactcc) to anneal specifically at the 5′ end of HA5, and a reverse primer (SEQ ID NO:2; agctctggtgaaggtctttctc) that can anneal both at the 3′end of HA5 and the 3′end of HA3 based on the sequence similarity between HA5 and HA3.

In the presence of a GSTT1 active allele, amplification occurs using the forward primer annealed at the 5′ end of HA5 and the reverse primer annealed at the 3′ end of HA5. In this case amplification using the reverse primer attached at the 3′end of HA3 cannot be accomplished since the primers are located 54 kb apart. If a GSTT1 deleted allele is present amplification with a reverse primer annealed at the 3′ end of HA5 is not possible since the recombination between both repetitive elements leads to the deletion of this fragment. However, since the reverse primer can also anneal at the 3′ end of HA3, PCR amplification using the forward primer at the 5′ end of HA5 and the reverse primer at the 3′ end of HA3 can take place. Amplifications of either the active or the deleted GSTT1 alleles using the aforementioned primers yield a 709 basepairs (bp) long PCR product. To differentiate between both alleles a restriction enzyme is used to cleave the PCR products in one or two sites. Since the same pair of primers is used to amplify the active and deleted GSTT1 alleles, failure in amplification will not yield a PCR product and cannot confound the genotyping of the sample. Furthermore, a constant site for the restriction enzyme is present in both alleles generating a constant 483 bp fragment, which serves as an internal control for enzyme digestion. An additional restriction site is present only in the GSTT1 active allele due to a G to A change in the 3′ end of HA5 compared to the 3′ end of HA3. In the presence of an active GSTT1 allele the 221 bp long fragment generated by digestion of the constant site is further digested in two fragments (127 and 94 bp).

PCR amplification was performed in a 22 μl total volume containing approximately 0.01 μg of genomic or tumor DNA, 10× buffer, 0.2 mM of each dNTP, Q solution (Qiagen, Valencia, Calif.), 5 pmol of the primers, dimethyl sulfoxide and 0.25U Taq polymerase. Initial denaturation at 95° C. for 5 min was followed by 34 cycles of denaturation at 95° C. for 1 min, annealing at 55° C. for 1 min, and extension at 72° C. for 1 min. The final extension was carried out at 72° C. for 7 min. PCR products were digested with 0.5 μl of HpyCH4IV and added to 10 μl of amplified sample diluted in 34.5 μl of distilled water and 5 μl of New England Buffer 1 (New England Biolabs, Beverly, Mass.). The products were electrophoresed on an 8% Sequagel acrylamide gel for 1 hour at 260V and stained with ethidium bromide prior to being visualized with ultraviolet radiation.

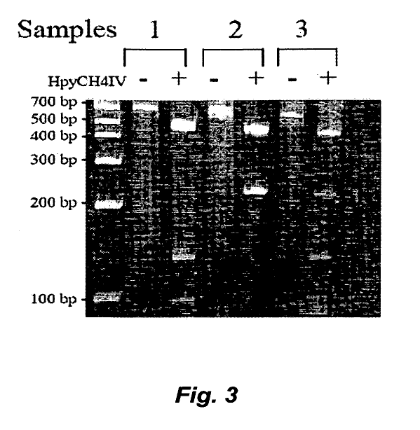

An example of the identification of the three possible GSTT1 genotypes is shown in FIG. 3. Advantages of the new method over existing methods include:

1. the ability to rapidly determine all three possible GSTT1 genotypes (homozygous active, heterozygous, and homozygous deleted.)

2. the presence of internal controls for both steps of the process (PCR amplification and restriction digestion.)

### Example 2

### Applying the New GSTT1 PCR Assay to a Group of NF2 Patients

We applied the PCR-based GSTT1 assay described in Example 1 to DNA samples from a subset of the patients currently enrolled in a natural history study of NF2 patients. The study examined NF2 patients primarily from the House Ear Institute (HEI, Los Angeles, Calif.), Massachusetts General Hospital (MGH, Boston, Mass.), the St. Mary's Hospital (United Kingdom) and Klinikum Nord Institute (Germany). The entry criteria were that the individuals were NF2 patients with confirmed diagnosis of NF2 since 1993 and presence of intracranial and/or spinal tumors. A total of 88 patients with 20 years of age or older were enrolled in this study, including 48 HEI patients and 13 MGH patients. At entry, each patient donated a small blood sample to the study for definition (or confirmation) of the underlying genomic change in the NF2 gene. Subsequently, each patient underwent yearly evaluation which includes cranial MRI, spinal MRI, ophthalmologic examination, audiometric exam, and general medical evaluation.

DNA from a subset of 31 of the 88 patients was examined. The results of applying the GSTT1 assay to these DNA samples showed the following:

1. Adequate amplification for rapid allelotyping was possible from archived genomic DNA in all cases.

2. Nine patients were homozygous active (+/+) for GSTT1, 16 were heterozygous (+/−) and six were homozygous deleted (−/−). This distribution is not significantly different between NF2 patients with meningiomas compared NF2 patients without meningiomas and confirms that GSTT1 alleles are not in disequilibrium with NF2 alterations themselves.

3. The natural history study offers a unique opportunity to study asymptomatic tumors since all patients underwent complete cranial MRI scan regardless of whether they had symptoms that might be referable to a meningioma. Amongst these 31 patients, 18 had no intra cranial meningiomas while the remainder had an average of 2.46 tumors (range 1 to 4). Of those with meningiomas, the GSTT1 haplotypes were four +/+, seven +/−, and two −/−, while of those without, five were +/+, nine +/− and four were −/− (Table 2). Although these numbers remain small, they support the hypothesis that GSTT1 status does not predispose to meningioma initiation. The average number of tumors per patient was 3 for patients +/+, 2 for +/−, and 3 for −/−.

TABLE 2
GSTT1 null genotype and tumor number
and volume in NF2 patients.

| | | | | Avg. | Avg. | |
|---|---|---|---|---|---|---|
| | | Patients | Patients | number of | tumor | |
| | Over- | with | without | tumors/ | volume/ | |
| Genotypes | all | meningiomas | meningiomas | patient | patient | |
| +/+ (n = 9) | 29% | 31% (n = 4) | 28% (n = 5) | 3.0 | 12.4 | cc |
| +/− (n = 16) | 52% | 54% (n = 7) | 50% (n = 9) | 2.0 | 3.58 | cc |
| −/− (n = 6) | 19% | 15% (n = 2) | 22% (n = 4) | 3.0 | 29.93 | cc |

Distribution of NF2 patients according to their GSTT1 status does not varies significantly between patients with and without meningiomas. However, in this small group of NF2 patients, total intracranial meningioma volume was found to be increased among NF2 patients carrying the null GSTT1 genotype. Abbreviations: Avg, average; +/+, homozygous active; +/−, heterozygous; −/−, null.

4. Amongst the 13 patients with meningiomas, we then reasoned that an indicator of tumor growth would be total intracranial volume of tumor. Total volume in patients with +/+ was 12.4 cc, +/− was 3.58 cc, and −/− was 29.93 cc. This exciting result suggests that GSTT1 null genotype is associated with meningioma tumor growth; additional experiments with a greater number of patients will be conducted to confirm these results. In addition, we recognize that half of the heterozygotes will become null during NF2 LOH and through LOH studies of tumors we are currently working to define the difference between heterozygotes with the null GSTT1 allele in cis and those with the null allele in trans.

These preliminary results show that in NF2 patients GSTT1 is not involved in meningioma initiation, but seems to lead to faster tumor growth and progression. The fact that meningiomas in NF2 patients are almost always low grade is due to the severity of this disease, which has an actuarial survival rate of 15 years after diagnosis, which does not allow enough time for meningioma progression.

Thus, analysis of GSTT1 status provides a valuable diagnostic and prognostic tool for NF2 patients, determining the potential for tumor growth based on GSTT1 status. Combined with a better understanding of the carcinogens inactivated by GSTT1 our results can suggest a change in lifestyle for NF2 patients carrying a GSTT1 null genotype may be helpful in reducing tumor growth and progression, and recommend early intervention for intracranial tumors.

### Example 3

### Determining GSTT1 Status in Sporadic Meningioma Patients

Using the PCR-based assay described in Example 1, we determined the GSTT1 status in 58 of our group of 62 patients informative at the NF2 locus. Twenty-one percent (12/58) of the patients had a null genotype, while 52% (30/58) and 27% (16/58) were heterozygous and homozygous for the GSTT1 active allele, respectively. It is interesting to note that the Hardy-Weinberg equilibrium is not observed, with the heterozygous genotype slightly over-represented. Our results corroborate a previous report of 270 Swedish individuals from a control group in which the GSTT1 null genotype was found in 10% of the population (Warholm, Rane et al. 1995). In this report additional phenotypic analysis of the samples showed that 50% of the samples were partial-conjugators (instead of the expected 40%) and 40% of the samples were full-conjugators (instead of the expected 50%). Frequency of GSTT1 null genotype among our sporadic meningioma patients (21%) was found to be higher that the expected 15-18% reported for control groups in two studies involving institutions from the New England area (Nelson, Wiencke et al. 1995; De Roos, Rothman et al. 2003). Even more interesting was the increased frequency of GSTT1 null genotype among patients who became symptomatic (progressed) for their meningiomas before 50 years of age (4 out of 17, 23.5%) and patients who became symptomatic (progressed) after 50 years of age (8 out of 41, 19.5%).

### Example 4

### GSTT1 LOH and Tumor Progression

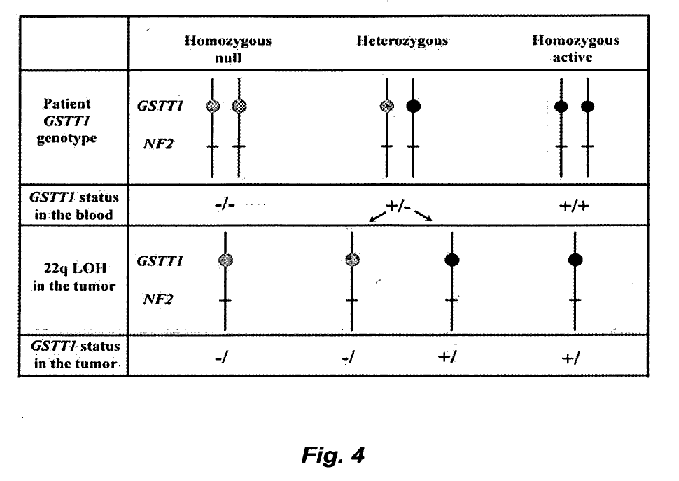

Monosomy of chromosome 22 will lead to loss of the GSTT1 allele located cis to the wild-type NF2 allele. Because patients carrying the null genotype have no GSTT1 activity, loss of heterozygosity (LOH) of 22q will not cause additional loss of enzymatic activity in the tumor. However, patients found to be homozygous for the GSTT1 active allele in the genomic DNA and showing LOH of chromosome 22 will always lose one active copy of GSTT1 in the tumor tissue independently of the position of the alleles. This loss can be predicted to cause an approximately 50% decrease in GSTT1 activity in the tumor tissue. In heterozygous patients, additional loss of GSTT1 alleles due to 22q LOH will occur trans to the initial NF2 inactivation event. Assuming that these events arise randomly, patients heterozygous for the GSTT1 deletion and showing LOH of 22q are expected to lose the remaining active GSTT1 allele in 50% of the cases, while 50% will lose the deleted allele. The former group of patients will consequently have no GSTT1 activity in the tumor tissue (FIG. 4).

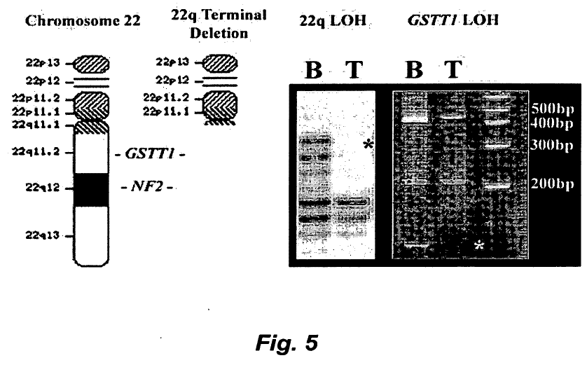

To determine the effect of GSTT1 LOH in our group of tumors we analyzed the GSTT1 status in the tumor DNA from 29 out of the 30 patients from our group of sporadic meningiomas found to be heterozygous for the GSTT1 deletion (in Example 3). These samples are characterized by the presence of four bands on the polyacrylamide gel used to genotype GSTT1 (483 bp, 221 bp, 127 bp, and 94 bp). Tumor DNA used to demonstrate 22q LOH was also used to determine GSTT1 genotype with the previously described PCR-based assay. Results obtained from the microarray comparative genomic hybridization (CGH) analysis of chromosome 22 were used for comparison. Eight tumors (28%) did not have 22q LOH, and carried the same heterozygous genotype present in genomic DNA. In the remaining 21 tumors, LOH of 22q caused additional loss of the GSTT1 active allele in 33% of the tumors, while 67% of them retained the active allele, with loss of the already deleted GSTT1 allele. Although we have a small sample size, our preliminary data suggests that loss of the active GSTT1 allele in heterozygous patients will lead faster tumor growth and progression (FIG. 5). Frequency of GSTT1 genotypes in the tumors also showed that 22q LOH leads to decreased number of active alleles among tumors, with the null genotype present in 40% of the tumors, while only 7% of the samples retained a homozygous active genotype in the meningiomas after LOH analysis (Table 3).

TABLE 3
Distribution of GSTT1 genotypes among sporadic meningiomas.

| | | Tumor after 22q |
|---|---|---|
| GSTT1 genotype | Blood (n = 58) | LOH (n = 57) |
| Homozygous active | 27% | 7% |
| Heterozygous | 52% | 56% |
| Homozygous deleted | 21% | 37% |

Genotyping of GSTT1 in our group of sporadic meningioma patients showed that 21% of them carry the null genotype of GSTT1 compared 15-18% frequency of the null genotype previously reported for individuals from the New England area (Nelson, Wiencke et al. 1995; De Roos, Rothman et al. 2003). Interestingly, analysis of the GSTT1 genotype in the tumors shows that 22q LOH leads to lack of GSTT1 activity in the tumor in 37% of the cases.

### REFERENCES

Abdel-Rahman, S. Z., W. A. Anwar, et al. (1998). “GSTM1 and GSTT1 genes are potential risk modifiers for bladder cancer.” Cancer Detect Prev 22(2): 129-38.
Al-Mefty, O., P. A. Kadri, et al. (2004). “Malignant progression in meningioma: documentation of a series and analysis of cytogenetic findings.” J Neurosurz 101(2): 210-8.
Arslantas, A., S. Artan, et al. (2002). “Comparative genomic hybridization analysis of genomic alterations in benign, atypical and anaplastic meningiomas.” Acta Neurol Beli 102(2): 53-62.
Arslantas, A., S. Artan, et al. (2003). “Detection of chromosomal imbalances in spinal meningiomas by comparative genomic hybridization.” Neurol Med Chir (Tokyo) 43(1): 12-8; discussion 19.
Baser, M. E., L. Kuramoto, et al. (2004). “Genotype-phenotype correlations for nervous system tumors in neurofibromatosis 2: a population-based study.” Am J Hum Genet 75(2): 231-9.
Bostrom, J., B. Meyer-Puttlitz, et al. (2001). “Alterations of the tumor suppressor genes CDKN2A (p16(INK4a)), p14(ARF), CDKN2B (p15(INK4b)), and CDKN2C (p18(INK4c)) in atypical and anaplastic meningiomas.” Am J Pathol 159(2): 661-9.
Brockmoller, J., I. Cascorbi, et al. (1996). “Combined analysis of inherited polymorphisms in arylamine N-acetyltransferase 2, glutathione S-transferases M1 and T1, microsomal epoxide hydrolase, and cytochrome P450 enzymes as modulators of bladder cancer risk.” Cancer Res 56(17): 3915-25.
Chen, K., Q. T. Jiang, et al. (2005). “Relationship between metabolic enzyme polymorphism and colorectal cancer.” World J Gastroenterol 11(3): 331-5.
De Roos, A. J., N. Rothman, et al. (2003). “Genetic polymorphisms in GSTMI, -P1, -T1, and CYP2E1 and the risk of adult brain tumors.” Cancer Epidemiol Biomarkers Prev 12(1): 14-22.
DeAngelis, L. M. (2001). “Brain tumors.” N Engl J Med 344(2): 114-23.
Deng, Z. L., Y. P. Wei, et al. (2005). “Polymorphism of glutathione S-transferase mu 1 and theta 1 genes and hepatocellular carcinoma in southern Guangxi, China.” World J Gastroenterol 11(2): 272-4.
Elexpuru-Camiruaga, J., N. Buxton, et al. (1995). “Susceptibility to astrocytoma and meningioma: influence of allelism at glutathione S-transferase (GSTT1 and GSTM1) and cytochrome P-450 (CYP2D6) loci.” Cancer Res 55(19): 4237-9.
Evans, D. G., S. M. Huson, et al. (1992). “A clinical study of type 2 neurofibromatosis.” Q J Med 84(304): 603-18.
Evans, D. G., L. Trueman, et al. (1998). “Genotype/phenotype correlations in type 2 neurofibromatosis (NF2): evidence for more severe disease associated with truncating mutations.” J Med Genet 35(6): 450-5.
Ezer, R., M. Alonso, et al. (2002). “Identification of glutathione S-transferase (GST) polymorphisms in brain tumors and association with susceptibility to pediatric astrocytomas.” J Neurooncol 59(2): 123-34.
Gutmann, D. H., J. Donahoe, et al. (2000). “Loss of DAL-1, a protein 4.1-related tumor suppressor, is an important early event in the pathogenesis of meningiomas.” Hum Mol Genet 9(10): 1495-500.
Hallier, E., T. Langhof, et al. (1993). “Polymorphism of glutathione conjugation of methyl bromide, ethylene oxide and dichloromethane in human blood: influence on the induction of sister chromatid exchanges (SCE) in lymphocytes.” Arch Toxicol 67(3): 173-8.
Jaaskelainen, J., M. Haltia, et al. (1986). “Atypical and anaplastic meningiomas: radiology, surgery, radiotherapy, and outcome.” Surg Neurol 25(3): 233-42.
Joachim, T., Z. Ram, et al. (2001). “Comparative analysis of the NF2, TP53, PTEN, KRAS, NRAS and HRAS genes in sporadic and radiation-induced human meningiomas.” Int J Cancer 94(2): 218-21.
Jourenkova-Mironova, N., A. Voho, et al. (1999). “Glutathione S-transferase GSTM1, GSTM3, GSTP1 and GSTT1 genotypes and the risk of smoking-related oral and pharyngeal cancers.” Int J Cancer 81(1): 44-8.
Khan, J., N. Z. Parsa, et al. (1998). “Detection of gains and losses in 18 meningiomas by comparative genomic hybridization.” Cancer Genet Cvtogenet 103(2): 95-100.
Kleihues, P., D. N. Louis, et al. (2002). “The WHO classification of tumors of the nervous system.” J Neuronathol Exp Neurol 61(3): 215-25; discussion 226-9.
Landi, S. (2000). “Mammalian class theta GST and differential susceptibility to carcinogens: a review.” Mutat Res 463(3): 247-83.
Leone, P. E., M. J. Bello, et al. (1999). “NF2 gene mutations and allelic status of 1p, 14q and 22q in sporadic meningiomas.” Oncozene 18(13): 2231-9.
Leuraud, P., E. Dezamis, et al. (2004). “Prognostic value of allelic losses and telomerase activity in meningiomas.” J Neurosurg 100(2): 303-9.
Leuraud, P., Y. Marie, et al. (2000). “Frequent loss of Ip32 region but no mutation of the p18 tumor suppressor gene in meningiomas.” J Neurooncol 50(3): 207-13.
Mautner, V. F., M. Lindenau, et al. (1996). “The neuroimaging and clinical spectrum of neurofibromatosis 2.” Neurosurgery 38(5): 880-5; discussion 885-6.
Nakamura, M. and M. Samii (2003). “Surgical management of a meningioma in the retrosellar region.” Acta Neurochir (Wien) 145(3): 215-9; discussion 219-20.
Nelson, H. H., J. K. Wiencke, et al. (1995). “Ethnic differences in the prevalence of the homozygous deleted genotype of glutathione S-transferase theta.” Carcinogenesis 16(5): 1243-5.
Nunes, F. and M. MacCollin (2003). “Neurofibromatosis 2 in the pediatric population.” J Child Neurol 18(10): 718-24.
Ozaki, S., T. Nishizaki, et al. (1999). “Comparative genomic hybridization analysis of genetic alterations associated with malignant progression of meningioma.” J Neurooncol 41(2): 167-74.
Parry, D. M., R. Eldridge, et al. (1994). “Neurofibromatosis 2 (NF2): clinical characteristics of 63 affected individuals and clinical evidence for heterogeneity.” Am J Med Genet 52(4): 450-61.
Pemble, S., K. R. Schroeder, et al. (1994). “Human glutathione S-transferase theta (GSTT1): cDNA cloning and the characterization of a genetic polymorphism.” Biochem J 300 (Pt 1): 271-6.
Perry, A., R. B. Jenkins, et al. (1996). “Cytogenetic analysis of aggressive meningiomas:

possible diagnostic and prognostic implications.” Cancer 77(12): 2567-73.
Ruttledge, M. H., J. Sarrazin, et al. (1994). “Evidence for the complete inactivation of the NF2 gene in the majority of sporadic meningiomas.” Nat Genet 6(2): 180-4.
Samii, M., G. A. Carvalho, et al. (1996). “Meningiomas of the tentorial notch: surgical anatomy and management.” J Neurosurg 84(3): 375-81.
Schroder, K. R., E. Hallier, et al. (1992). “Dissociation of a new glutathione S-transferase activity in human erythrocytes.” Biochem Pharmacol 43(8): 1671-4.
Sobti, R. C., A. I. Al-Badran, et al. (2005). “Genetic polymorphisms of CYP2D6, GSTM1, and GSTT1 genes and bladder cancer risk in North India.” Cancer Genet Cvtogenet 156(1): 68-73.
Sprenger, R., R. Schlagenhaufer, et al. (2000). “Characterization of the glutathione S-transferase GSTT1 deletion: discrimination of all genotypes by polymerase chain reaction indicates a trimodular genotype-phenotype correlation.” Pharmacogenetics 10(6): 557-65.
Srivastava, D. S., A. Mandhani, et al. (2005). “Genetic polymorphism of glutathione S-transferase genes (GSTM1, GSTT1 and GSTP1) and susceptibility to prostate cancer in Northern India.” BJU Int 95(1): 170-3.
Tsabouri, S., I. Georgiou, et al. (2004). “Glutathione sulfur transferase M1 and T1 genotypes in chronic lymphoblastic leukemia.” Hematol J 5(6): 500-4.
Warholm, M., A. Rane, et al. (1995). “Genotypic and phenotypic determination of polymorphic glutathione transferase T1 in a Swedish population.” Pharmacogenetics 5(4): 252-4.
Whittle, I. R., C. Smith, et al. (2004). “Meningiomas.” Lancet 363(9420): 1535-43.
Wrensch, M., K. T. Kelsey, et al. (2004). “Glutathione-S-transferase variants and adult glioma.” Cancer Epidemiol Biomarkers Prev 13(3): 461-7.

Other aspects of the invention will be clear to the skilled artisan and need not be repeated here. Each reference cited herein is incorporated by reference in its entirety.

The terms and expressions which have been employed are used as terms of description and not of limitation, and there is no intention in the use of such terms and expressions of excluding any equivalents of the features shown and described or portions thereof, it being recognized that various modifications are possible within the scope of the invention.

We claim:

1. A method for determining the genotype of GSTT1 in a subject, comprising:
amplifying fragment(s) of GSTT1 by subjecting a genomic DNA sample of the subject to DNA amplification using a 5′ primer and a 3′ primer that together amplify fragments of intact GSTT1 alleles and/or deleted GSTT1 alleles,
wherein the 5′ primer anneals to a first HA5 element sequence or to a DNA region located immediately adjacent and upstream (5′) of HA5,
wherein the 3′ primer anneals to a second HA5 element sequence and to a HA3 element sequence, and
wherein each of the second HA5 element sequence and the HA3 element sequence are spaced apart from and 3′ to the first HA5 element sequence.

2. The method of claim 1, wherein a fragment amplified by the 5′ primer and the 3′ primer annealed to the second HA5 element sequence is about the same length as a fragment amplified by the 5′ primer and the 3′ primer annealed to the HA3 element sequence.

3. The method of claim 1, further comprising digesting the amplified fragment(s) with a restriction enzyme that differentially cleaves the amplified fragment(s) depending on whether the 3′ primer annealed to the second HA5 element sequence or to the HA3 element sequence.

4. The method of claim 1, further comprising determining the size of the fragment(s) by gel electrophoresis.

5. The method of claim 3, further comprising determining the size of the fragment(s) by gel electrophoresis.

6. The method of claim 1, wherein the genomic DNA sample of the subject is obtained from blood or from a tumor.

7. The method of claim 1, wherein the 5′ primer comprises SEQ ID NO: 1.

8. The method of claim 1, wherein the 5′ primer consists of SEQ ID NO: 1.

9. The method of claim 1, wherein the 3′ primer comprises SEQ ID NO:2.

10. The method of claim 1, wherein the 3′ primer consists of SEQ ID NO:2.

11. The method of claim 1, wherein the DNA amplification is polymerase chain reaction (PCR).

12. The method of claim 1, wherein the 5′ primer is up to about 1 kb in the 5′ direction from the HA5 element.

13. The method of claim 12, wherein the 5′ primer is up to about 1 kb in the 5′ direction from SEQ ID NO: 1.

14. A kit for genotyping GSTT1, comprising:
a first container containing a 5′ primer, wherein the 5′ primer anneals to a first HA5 element sequence, and
a second container containing a 3′ primer, wherein the 3′ primer anneals to a second HA5 element sequence and to a HA3 element sequence,
wherein each of the second HA5 element sequence and the HA3 element sequence are spaced apart from and 3′ to the first HA5 element sequence, and
wherein the 5′ primer and the 3′ primer together amplify fragments of intact GSTT1 alleles and/or deleted GSTT1 alleles in a DNA amplification reaction.

15. The kit of claim 14, further comprising a third container containing a DNA restriction enzyme that differentially cleaves the amplified fragment(s) depending on whether the 3′ primer annealed to the second HA5 element sequence or to the HA3 element sequence.

16. The kit of claim 14, further comprising one or more containers containing buffer solution(s), DNA polymerase enzyme(s), restriction enzyme(s) and/or nucleotide solution(s).

17. The kit of claim 16, wherein the DNA polymerase enzyme is a thermostable DNA polymerase.

18. The kit of claim 14, further comprising one or more containers containing GSTT1 control DNA.

19. The kit of claim 18, wherein the GSTT1 DNA is homozygous for GSTT1 wild type alleles, homozygous for GSTT1 null alleles and/or heterozygous for GSTT1 wild type and null alleles.

20. The kit of claim 14, wherein the 5′ primer is SEQ ID NO:1.

21. The kit of claim 14, wherein the 3′ primer is SEQ ID NO:2.

22. The kit of claim 14, wherein the 5′ primer is SEQ ID NO:1 and wherein the 3′ primer is SEQ ID NO:2.

23. A method for determining tumor growth and progression in a subject, comprising
obtaining a genomic DNA sample from the subject, and
determining the genotype of GSTT1 in the genomic DNA sample according to claim 1,
wherein a GSTT1 null genotype indicates that the subject has or will have elevated tumor growth and progression.

24. The method of claim 23, wherein the tumor is one or more meningiomas.

25. The method of claim 23, wherein the tumor is one or more bladder cancers, squamous cell carcinomas, cancers in the upper aero digestive tract, gastric cancers, acute lymphoblastic leukaemias, hepatocellular carcinomas, cervical cancers, breast cancers, lung cancers, acute myeloid leukemias, thyroid cancers, astrocytomas, prostate cancers, hepatocellular carcinomas, colon cancers, bladder cancers or chronic lymphoblastic leukemias.

26. The method of claim 23, wherein the subject has or is suspected of having neurofibromatosis 2 (NF2).

27. A method for determining prognosis of a subject, comprising
determining a GSTT1 genotype of the subject according to claim 1,
wherein a homozygous null GSTT1 genotype or a heterozygous GSTT1 genotype is indicative of a relatively poor prognosis for the subject, and wherein a homozygous wild type GSTT1 genotype is indicative of a relatively good prognosis for the subject.

28. The method of claim 27, wherein the subject has or is suspected of having one or more meningiomas.

29. The method of claim 27, wherein the subject has or is suspected of having one or more bladder cancers, squamous cell carcinomas, cancers in the upper aero digestive tract, gastric cancers, acute lymphoblastic leukaemias, hepatocellular carcinomas, cervical cancers, breast cancers, lung cancers, acute myeloid leukemias, thyroid cancers, astrocytomas, prostate cancers, hepatocellular carcinomas, colon cancers, bladder cancers or chronic lymphoblastic leukemias.

30. The method of claim 27, wherein the subject has or is suspected of having neurofibromatosis 2 (NF2).

31. A method for determining the suitability of therapeutic intervention for a patient having or suspected of having one or more meningiomas, comprising
determining a GSTT1 genotype of the subject according to claim 1,
wherein a homozygous null GSTT1 genotype indicates that therapeutic intervention is suitable.

32. The method of claim 31, wherein the therapeutic intervention is GST replacement therapy.

33. The method of claim 31, wherein the GST replacement therapy comprises administration of an effective amount of GSTT1 to the patient.

34. The method of claim 33, wherein the therapeutic intervention is surgery.

35. The method of claim 31, wherein the meningioma patients are NF2 patients.

36. The method of claim 31, wherein the meningiomas are intracranial meningiomas.
